(19) United States
(12) Patent Application Publication
(10) Pub. No.: US 2026/0297553 A1
(43) Pub. Date: Oct. 1, 2026

(54) Improved Enzyme Sensor Membrane for Lactose

(71) Applicant: YSI, Inc., Yellow Springs, OH (US)

(72) Inventors: Kevin Schlueter, New Carlisle, OH (US); Kristopher Kleski, Columbus, OH (US); Marcel Madaras, Mason, OH (US)

(21) Appl. No.: 19/630,816

(22) Filed: Mar. 27, 2026

Related U.S. Application Data
(60) Provisional application No. 63/780,845, filed on Mar. 31, 2025.

(51) Int. Cl. C12N 11/18; C12N 9/04; C12N 9/38; C12N 11/089; C12N 11/12

(52) U.S. Cl. CPC C12N 11/18; C12N 9/0006; C12N 9/2471; C12N 11/089; C12N 11/12; C12Y 101/03004; C12Y 302/01023

(57) ABSTRACT

A trilaminar membrane invention to quantitate lactose in conjunction with a potentiostatic amperometric cell. The invention features a support layer, an immobilized enzyme layer, and an electrode-facing exclusion layer. The support layer is a homogeneous, semi-permeable polycarbonate film that controls diffusion rates of analyte into the enzyme reaction layer. The analyte is then oxidatively processed to generate hydrogen peroxide (H₂O₂) by the immobilized 2-enzyme system comprised of β-galactosidase and glucose oxidase. Hydrogen peroxide then diffuses through the permselective exclusion layer to react at a poised sense electrode probe to generate an electrical signal for analyte quantitation. The thin sensing membrane is affixed to an O-ring for mounting on the face of the electrode probe.

### CROSS REFERENCE TO RELATED APPLICATIONS

The application claims benefit to U.S. patent application Ser. No. 63/780,845, filed 31 Mar. 2025, which is hereby incorporated by reference.

The application relates to and builds on technology developed by the assignee (i.e., The Yellow Springs Instrument Co., Inc., hereinafter known as “YSI”) of the instant application and disclosed in the following U.S. Pat. Nos. 3,539,455; 3,979,274; 4,073,713; 4,220,503; 4,356,074; 5,520,788; 5,766,839 and 6,020,052, which are all hereby incorporated by reference in their entirety.

### BACKGROUND OF THE INVENTION

Amperometric systems have become the gold standard for clinical glucose quantitative analysis due to the efficiency and accuracy of the method. Seminal work has an overwhelming focus on clinical glucose quantitation, which has laid the foundation for the development of similar methods designed to quantify other carbohydrate analytes, such as lactose. These carbohydrate analytes are inherently amperometrically inactive, which dictates these analytes must be chemically processed to produce an amperometrically active species. The common strategy to fulfill this requirement is to expose analyte to an oxidative enzyme—for which the analyte is a substrate. For example, the reaction illustrated below is utilized to generate the amperometrically active hydrogen peroxide from lactose.

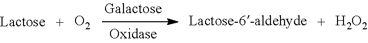

The hydrogen peroxide generated in this reaction can then be measured in an amperometric cell such as the system disclosed in YSI's U.S. Pat. No. 3,539,455.

The current flowing at the sensing electrode is proportional to the amount of hydrogen peroxide produced; in turn, lactose can be indirectly measured. One of the major benefits to this strategy is the use of a catalytic enzyme system, which significantly limits the number of chemical species able to produce a positive signal. The method is further refined using a semi-permeable, outer support layer of polycarbonate film which restricts high molecular weight species such as proteins and other macromolecules from diffusing into the enzyme layer. YSI's U.S. Pat. No. 5,520,788 discloses examples of such support layers. This analyte facing layer also limits the diffusion of analyte as compared to dissolved oxygen. This is especially important in native samples that may contain excess analyte as compared to oxygen, and therefore oxygen is the rate limiting reagent. In such cases, linearity is reduced to a small and unfavorable window that requires sample dilution to empirically derived linear ranges. Combined, the enzyme system and outer support layer allow for the use of native, unaltered samples such as milk and fermented milk products to be analyzed. After the enzymatic production of hydrogen peroxide via analyte oxidation, the hydrogen peroxide diffuses through a third layer before interacting with the electrode surface. This third layer acts as another exclusion layer, but with a much lower pore size so as to permit diffusion of chemical species less than approximately 180 Dalton. YSI's U.S. Pat. No. 5,766,839 discloses in detail examples such barrier layers. This restrictive diffusion prevents oxidizable species such as acetaminophen (Paracetamol, Tylenol®), ascorbic acid and uric acid from producing false positive signals.

Indeed, this trilaminar design, as disclosed in YSI's U.S. Pat. No. 3,979,274 and YSI's U.S. Pat. No. 4,073,713, has led to successful commercial methods for quantitating analytes in complex matrices. Such combinations of enzyme membranes and amperometric cells are described as enzyme electrodes. One such enzyme electrode immobilizes, through the use of chemical crosslinking, galactose oxidase between the outer support layer and the inner electrode-facing exclusion layer. These galactose oxidase membranes are intended to measure analytes such as galactose and lactose. However, the galactose oxidase enzyme exhibits relatively low activity and stability and requires activation with redox materials such as ferricyanide and stabilizers (e.g., cupric ion) during the immobilization step. YSI's U.S. Pat. No. 4,220,503 discloses an example of activation and stabilization of this enzyme. Galactose oxidase also has a relatively high promiscuity and can oxidize several substrates, most of which may simultaneously exist in the same native sample. Examples consist of galactose, lactose, glycerol, glyceraldehyde, and dihydroxyacetone. Unfortunately, under normal conditions galactose oxidase enzyme electrodes cannot discriminate multiple substrates in the same sample and therefore cannot provide an accurate response to the desired analyte. This limitation requires separation of the desired analyte from complex matrices or at least a complete knowledge of the sample matrix. An elegant strategy to control galactose specificity was to apply a redox potential to the enzyme layer, e.g., as disclosed in YSI's U.S. Pat. No. 4,356,074. However, this strategy has its own practical limitations. Existing galactose oxidase electrodes are operated in such a way that operators have a complete knowledge of sample matrix to ensure that cross reactivity does not provide false positive scenarios.

In effect, current sensing membranes use an expensive custom galactose oxidase enzyme for lactose sensing and sometimes fail production quality standards. Because of this, there is a need for a better sensor membrane for quantifying lactose in a liquid sample.

### SUMMARY OF THE INVENTION

In summary, the present invention provides a trilaminar membrane configured to quantitate lactose in conjunction with an amperometric cell. The trilaminar membrane features a support layer, an immobilized enzyme reaction layer, and a permselective electrode-facing exclusion layer, which combined to form part of a lactose enzyme electrode. The outer support layer may be a homogeneous, semi-permeable polycarbonate film configured to control diffusion rates of an analyte into the immobilized enzyme reaction layer. The analyte is then oxidatively processed to generate hydrogen peroxide by a new and unique immobilized 2-enzyme system comprising two enzymes: β-galactosidase and glucose oxidase. The hydrogen peroxide produced then diffuses through the permselective electrode-facing exclusion layer to react at a poised sense electrode to generate a signal containing information for analyte quantitation. The trilaminar membrane is a thin sensing membrane that may be affixed to an O-ring for mounting on the face of a lactose enzyme electrode probe inserted into a sample module of a biochemistry analyzer, e.g., including YSI's 2700 Series or 2900 Series Biochemistry Analyzers.

In effect, the present invention provides a technique in which the lactose enzyme electrode can be manufactured without the use of galactose oxidase and therefore without the use of activating agents and stabilizing agents. These two enzymes consisting of β-galactosidase and glucose oxidase both exhibit relatively high stability and sensitivity. Specificity is also improved by reducing the pool of cross-reactive species that promote false positive responses to two species: lactose and glucose. The 2-enzyme system generates amperometrically active hydrogen peroxide (H₂O₂) through an initial, magnesium-ion-dependent cleavage of the glycosidic bond of lactose to generate galactose and glucose, e.g., using the reaction illustrated below:

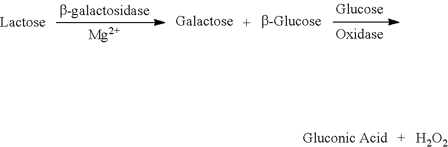

This enzymatic cleavage of lactose conserves the anomeric configuration of the glucose moiety, which is approximately 60% beta-anomer when lactose is at equilibrium. Therefore, an enzyme such as mutarotase is not required for this reaction to proceed. In the above reaction, the β-glucose is then oxidized by glucose oxidase to generate the gluconic acid and hydrogen peroxide (H₂O₂).

It is expected that some native samples may contain lactose as well as glucose. Samples that contain glucose directly affect the 2-enzyme electrode to provide inflated lactose signals due to glucose oxidase being the oxidative enzyme that produces the electrochemically active hydrogen peroxide. To account for these false positive scenarios, the 2-enzyme electrode system according to the present invention may be coupled to a separate glucose enzyme electrode of the same amperometric cell. Briefly, the signal generated from the glucose enzyme electrode can be subtracted from the signal generated from the 2-enzyme electrode to produce a net lactose signal from lactose alone. Thus, this invention represents a lactose enzyme electrode that does not require the use of stabilizing agents and improves analyte specificity as compared to the known lactose sensing-galactose oxidase enzyme electrodes.

By way of example, the semi-permeable outer support layer may consist of a polycarbonate film typically within 5-20 micron in thickness. Reasonably small pore sizes of 200 Å or less and pore densities on the order of 6×10⁸ through 6×10⁹/cm² are usually considered for this application to control analyte diffusion rates. These pores may be created in the polycarbonate film, e.g., by the process of particle beam track etching. The pore size and number density are adjusted to the application optimum during the track-etch process.

By way of further example, the electrode-facing exclusion layer may be made from a two-component cellulose acetate-cellulose acetate butyrate mixture dissolved in minimal amount of high-volatile, low-boiling point (<200° C.) solvent such as nitromethane to form a resin. This resin may also contain a plasticizing agent of higher boiling point as compared to the solvent, preferably exceeding 80° C. The resulting film may be ideally less than 2 microns in thickness with small intra-matrix pores that exclude species of greater than ~180 Daltons from diffusing.

By way of still further example, the immobilized 2-enzyme layer may act as a signal generator, a barrier, and an adhesive layer for the trilaminar design. Signal generation occurs as previously described when in contact with analyte containing solutions. The process of immobilization inherently generates adhesive properties in the center layer of the trilaminar design which maintains the invention's integrity. This immobilization may be achieved, e.g., by a treatment with a chemical protein crosslinker such as glutaraldehyde or any of many others recognized by those familiar with the art. This structural integrity may be further enhanced, e.g., by affixing the trilaminar membrane to a silicone O-ring, e.g., which also serves to create a water-tight seal between the lactose enzyme electrode and the sample well and keeps the reaction well contents separated from the electrode face. Thirdly, the immobilized 2-enzyme layer acts as another semi-permeable barrier, in which pore size is a function of buffer salt concentration during immobilization. Buffers of increasing ionic strength will generate increased intra-enzyme layer pore sizes and increased pore density as disclosed in YSI's U.S. Pat. No. 6,020,052. Due to this phenomenon, immobilization buffers typically do not exceed 0.1 M buffer salts.

In particular, the operation of the enzyme electrodes involves coupling the trilaminar membrane to a platinum electrode with an Ag/AgCl reference electrode, e.g., where both electrodes are coupled to a sample chamber or module of a biochemistry analyzer. Running buffer should typically be phosphate-based physiological saline buffer, such as YSI's 2357 buffer. For example, see User's Manual for YSI's Glucose & L-Lactate Analyzer, as well as YSI 2900 Series Biochemistry Analyzers, Operations and Maintenance Manual, which are both hereby incorporated by reference in its entirety. Due to the dependence of the magnesium ion in the glycosidic cleavage step, a magnesium cofactor preferably should also be supplemented to the running buffer. This dependance has been shown, where magnesium ion containing buffer provided an initial robust signal generation in the presence of analyte. However, this signal was drastically reduced with the removal of magnesium ion from the running buffer. The signal was then subsequently reactivated when the enzyme electrode was again exposed to a magnesium ion containing running buffer. FIG. 3 shows data that demonstrates that the enzyme electrode response is dependent on magnesium ion cofactor.

The lactose 2-enzyme electrode maintains a stable calibration signal as well as stable responses to analyte at concentrations near the upper limit of linearity over the course of multiple days as shown in FIGS. 4 and 5, respectively. The lactose 2-enzyme electrode also maintains a stable signal at lower concentrations, with a limit of quantitation at 0.06 g/L (data not shown).

Amperometric cells coupled with both lactose 2-enzyme electrodes and glucose enzyme electrodes can accurately quantify lactose in solutions containing both lactose and glucose. Simply, the signal generated by the glucose enzyme electrode may be subtracted from the lactose enzyme electrode to generate the net lactose signal. This performance has been demonstrated in solutions containing a constant lactose concentration of 0.5 g/L while increasing the amount of glucose from 1 g/L to 9 g/L. This dual chemistry performance is illustrated in FIG. 6, and it is readily observed that lactose signals remain stable and accurate up to approximately 6 g/L glucose. It is suspected that at high glucose concentrations, linearity is lost due to saturation of analyte in the enzyme layer.

The present invention provides a lactose 2-enzyme electrode system for use, e.g., on YSI's 2300, 2700 and 2900 Biochemistry Analyzers, to quantify lactose in solutions with complex matrices. The enzyme electrode can be manufactured without the use of redox activating reagents and stabilizers. Additionally, the enzyme electrode maintains a steady calibration signal over a working life of at least 10 days while maintaining signals in the upper and lower limits of linearity. The current galactose-oxidase based membranes are limited to five days working life. The present invention provides a method in which lactose can be quantified in the presence of galactose oxidase-cross reactive species such as glycerol without interference. In solutions containing both lactose and glucose, the present invention can be coupled with a glucose enzyme electrode to provide net lactose signals. The present invention provides a technique for the amperometric detection of lactose that is of significant commercial importance.

### Specific Embodiments

According to some embodiments, the present invention provides a new and unique laminated membrane to quantitate lactose in an analyte sample using a biochemistry analyzer.

The laminated membrane features a support layer, an exclusion layer and an enzyme reaction layer.

The support layer is configured to control a diffusion rate of an analyte sample having lactose into the laminated membrane.

The exclusion layer is configured to diffuse hydrogen peroxide (H₂O₂) to the sensing electrode, and rejecting unwanted interferent substances.

The enzyme reaction layer has a 2-enzyme system configured between the support layer and the exclusion layer. The 2-enzyme system includes β-galactosidase and glucose-oxidase configured to process oxidatively the analyte sample having the lactose and produce the hydrogen peroxide (H₂O₂) for diffusing through the exclusion layer and reacting with a sensor electrode to quantitate the lactose in the analyte sample with the biochemistry analyzer.

The laminated membrane may include one or more of the following features:

The β-galactosidase in the 2-enzyme system may be configured to process the analyte sample having the lactose into galactose and β-glucose, and the glucose-oxidase in the 2-enzyme system may be configured to process the β-glucose and forms the gluconic acid and hydrogen peroxide (H₂O₂).

The enzyme reaction layer may be configured as an immobilized 2-enzyme layer between the support layer and the exclusion layer acting as a signal generator, a barrier and an adhesive layer.

The 2-enzyme system may include the β-galactosidase and magnesium ions configured to process the lactose into galactose and β-glucose; and the 2-enzyme system is based upon a reaction, as the follows:

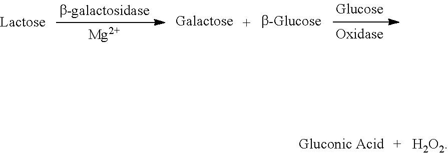

The support layer may be a homogeneous, semi-permeable polycarbonate film, where the homogeneous, semi-permeable polycarbonate film has a thickness of 5-20 microns, or pore sizes of 200 Å or less, or pore densities on an order of 6×10⁸ through 6×10⁹/cm², or some combination thereof.

The exclusion layer may be a film made from a two-component cellulose acetate-cellulose acetate-butyrate mixture, where the film has a thickness of 1 to 3 microns.

The laminated membrane may be configured in, and form part of, a lactose enzyme electrode.

The laminated membrane may include an O-ring affixed to an outer surface of the laminated membrane and configured to mount the laminated membrane on an electrode probe of the biochemistry analyzer.

### A Method

According to some embodiments, the present invention may take the form of a method to quantitate lactose in an analyte sample using a laminated membrane in a biochemistry analyzer.

The method features steps for:
configuring a laminated membrane with a support layer, an exclusion layer and an enzyme reaction layer having a 2-enzyme system with β-galactosidase and glucose and oxidase and being arranged between the support layer and the exclusion layer;
controlling with the support layer a diffusion rate of an analyte sample having lactose into the laminated membrane;
restricting interferences with the exclusion layer that also allows diffusion of hydrogen peroxide (H₂O₂) from the enzyme reaction layer; and
processing oxidatively with the 2-enzyme system having β-galactosidase and glucose-oxidase the analyte sample having the lactose and producing the hydrogen peroxide (H₂O₂) for diffusing through the exclusion layer and reacting with a sensor electrode to quantitate the lactose in the analyte sample using the laminated membrane in a biochemistry analyzer.

The method may also include one or more of the features set forth herein.

### Advantages

By way of example, some advantages of the present invention include the following: These two alternative enzymes are ~⅕ the cost of the current employed enzyme and are much more thermostable; the new sensor membranes are trilaminar and do not delaminate in production or near-term storage; additionally, these new sensor membranes are not co-reactive to glycerol or galactose like the current ones.

Other advantages are set forth below.

### BRIEF DESCRIPTION OF THE DRAWING

The drawing includes FIGS. 1-11, as follows:

FIG. 1 shows a trilaminar member having a 2-enzyme system, according to some embodiments of the present invention.

FIG. 2 shows a sample chamber or module, e.g., configured to receive a lactose 2 enzyme electrode with a trilaminar member therein and a reference electrode, according to some embodiments of the present invention.

FIG. 3 shows a graph of lactose calibration current (nA) vs. time (minutes) with and without a magnesium supplement in a buffer solution.

FIG. 4 shows a graph of an average calibration net plateau current (nA) vs. working life (days) of 6 lactose 2ES enzyme electrodes.

FIG. 5 shows a graph of an average response to a 25.0 g/L lactose standard (g/L) vs. working life (days) of 6 lactose 2ES enzyme electrodes

FIG. 6 shows a graph of an average response (g/L) of lactose (g/L) vs. glucose (g/L) of 6 lactose 2ES enzyme electrodes for solutions containing a constant concentration of 0.5 g/L lactose in relation to increasing glucose concentrations.

FIG. 7 shows a cutaway perspective view of YSI's 2900 Series BioChemistry Analyzer.

FIG. 8 shows YSI's 2900 Series Sample Module, e.g., which forms part of its 2900 Series BioChemistry Analyzer shown in FIG. 7.

FIG. 9 shows YSI's 2900 Series electrochemical probe having a trilaminar membrane according to the present invention that may be coupled to and form part of YSI's 2900 Series Sample Module shown in FIG. 8.

FIG. 10 is a photograph of a finished fabricated trilaminar sensing membrane on the right, and a face of an electrode probe on the left, according to some embodiments of the present invention, where the trilaminar member is gripped in a tweezers and mounted on a silicone rubber O-ring, and where the face of the electrode has a central platinum button sense electrode surrounded by a ring of dielectric potting that is surrounded by a wider band having an Ag/AgCl reference electrode and sealed to an outer plastic sleeve by the dielectric potting.

FIG. 11 shows an example of a fabrication process, according to some embodiments of the present invention.

### DETAILED DESCRIPTION OF THE PRESENT INVENTION

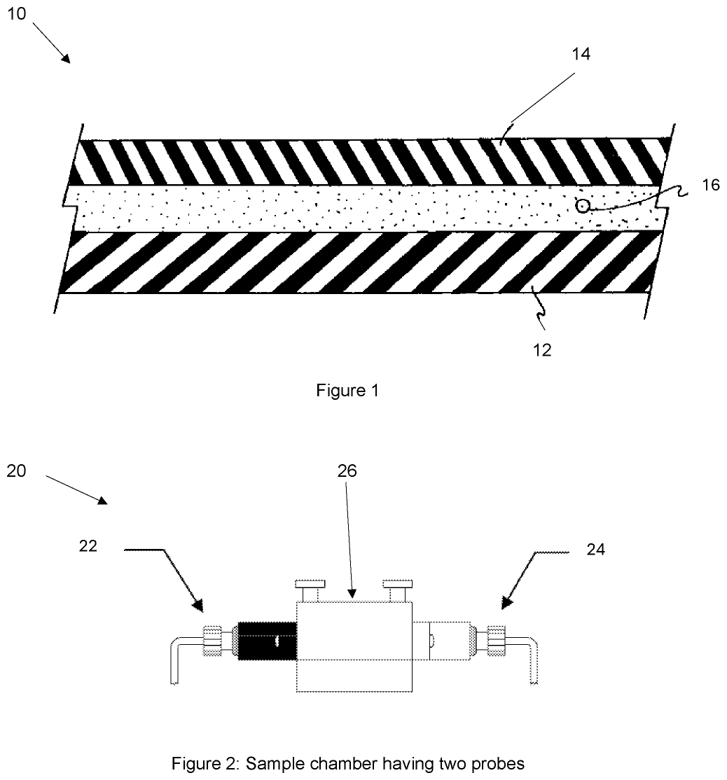

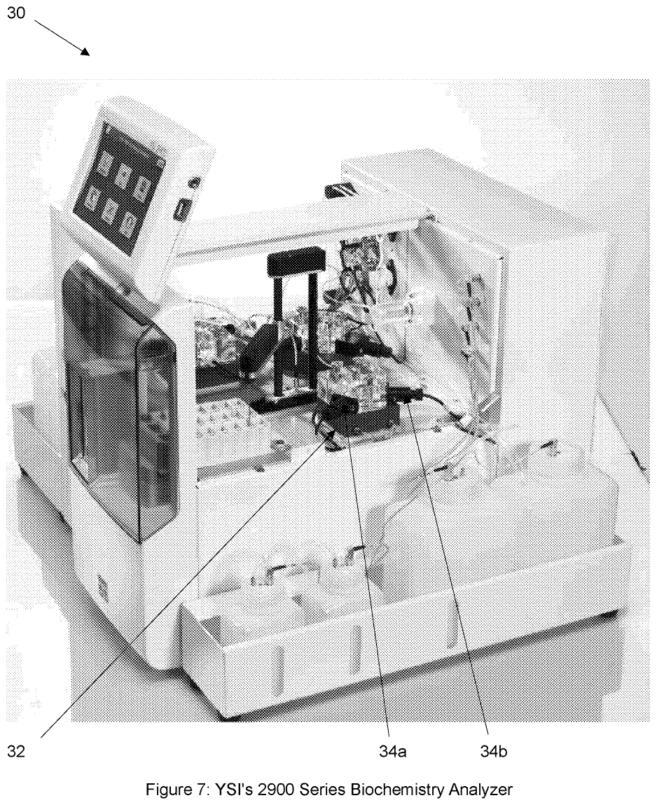

FIG. 1 shows a new and unique laminated membrane generally indicated as 10 to quantitate lactose in an analyte sample using a biochemistry analyzer 30, e.g., like that shown in FIG. 7.

The laminated membrane 10 is a trilaminar membrane having a support layer 12, an exclusion layer 14 and an enzyme reaction layer 16.

The support layer 12 is configured to control a diffusion rate of an analyte sample having lactose into the laminated membrane. By way of example, and consistent with that disclosed herein, the support layer 12 may be a homogeneous, semi-permeable polycarbonate film, including where the homogeneous, semi-permeable polycarbonate film has a thickness of 5-20 microns, or pore sizes of 200 Å or less, or pore densities on an order of 6×10⁸ through 6×10⁹/cm², or some combination thereof. The support layer 12 is configured to face a sample well and fluidically interact with the analyte sample having lactose contained in the sample well.

The exclusion layer 14 is configured to diffuse hydrogen peroxide (H₂O₂) from the enzyme reaction layer 16. By way of example, and consistent with that disclosed herein, the exclusion layer 14 may be configured as a film made from a two-component cellulose acetate-cellulose acetate-butyrate mixture, e.g., including where the film has a thickness of 1 to 3 microns. The exclusion layer 14 is configured to face a sensing electrode that measures lactose quantitatively.

The enzyme reaction layer 16 features a 2-enzyme system configured between the support layer 12 and the exclusion layer 14. The 2-enzyme system includes β-galactosidase and glucose oxidase configured to process oxidatively the analyte sample having the lactose and produce the hydrogen peroxide (H₂O₂) for diffusing through the exclusion layer 14 and from the laminated membrane 10 and reacting with a sensor electrode to quantitate the lactose in the analyte sample with the biochemistry analyzer 30 (FIG. 7).

The β-galactosidase in the 2-enzyme system may be configured to process the analyte sample having the lactose into galactose and β-glucose, and the glucose and oxidase in the 2-enzyme system may be configured to process the β-glucose and provide the gluconic acid and hydrogen peroxide (H₂O₂).

The enzyme reaction layer 16 may be configured as an immobilized 2-enzyme layer between the support layer 12 and the exclusion layer 14 acting as a signal generator, a barrier and an adhesive layer. Signal generation occurs as previously described when in contact with the analyte sample containing solutions. The process of immobilization inherently generates adhesive properties in the center enzyme reaction layer 16. The immobilization may be achieved, e.g., by a treatment with a chemical protein crosslinker such as glutaraldehyde or any of many others recognized by those familiar with the art. The immobilized 2-enzyme layer acts as another semi-permeable barrier, in which pore size is a function of buffer salt concentration during immobilization.

The 2-enzyme system may also include the β-galactosidase and a magnesium ion cofactor configured to process the lactose into galactose and β-glucose; and
the 2-enzyme system may be based upon a reaction, e.g., as follows:

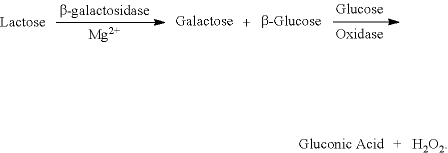

By way of example, FIG. 2 shows a sample chamber 20 having enzyme probes/electrodes 22, 24 and a sample well 26 for containing the analyte sample having the lactose therein. The laminated membrane 10 may be configured in, and form part of, one of the enzyme probes/electrodes 22, 24, e.g. as a lactose enzyme electrode according to the present invention. See also that shown and described in relation to FIGS. 7-9 below. By way of example, the other enzyme probes or electrodes may be a reference probe.

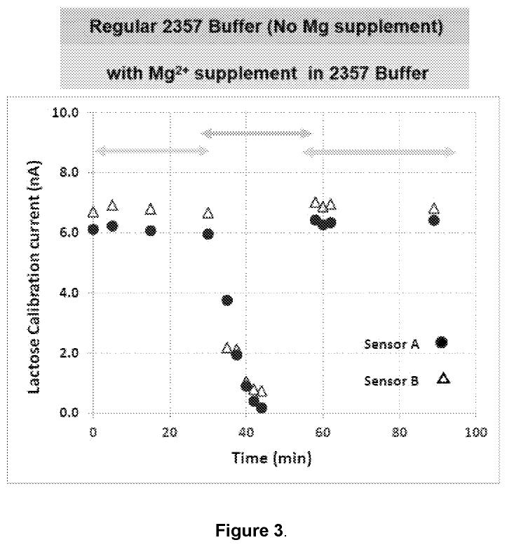

### FIGS. 3-6

FIG. 3 shows that the lactose 2ES enzyme electrode 10 is dependent on magnesium ion supplement in running buffer. Two lactose 2ES enzyme electrodes/sensors A and B showed an initial, stable calibration net plateau current between 6-7 nA in the presence of magnesium ion supplemented running buffer. This activity was completely reduced to 0 nA when magnesium ion was removed from running buffer. Activity was completely recovered when magnesium ion supplemented running buffer was restored.

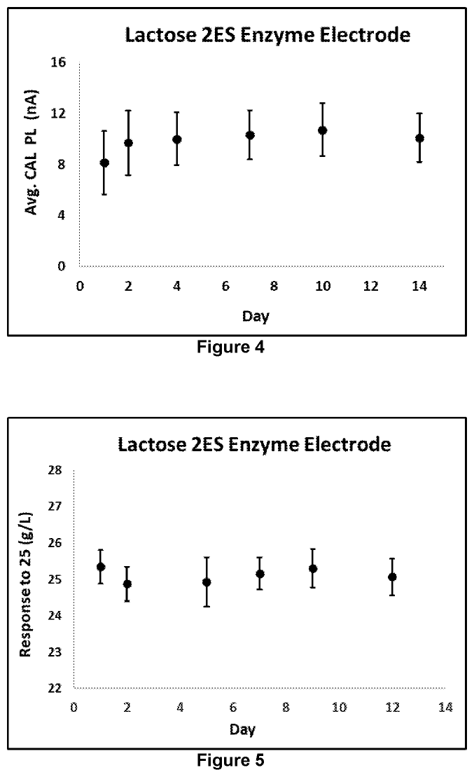

FIG. 4 shows an average calibration net plateau current vs working life of n=6 lactose 2ES enzyme electrodes. This data demonstrates the relative stability of lactose 2ES enzyme electrode sensitivity over the course of a 10-day working life where day 1 is the day of installation.

FIG. 5 shows an average response to 25.0 g/L lactose standard vs working life of n=6 lactose 2ES enzyme electrodes. This data demonstrates the relative stability of lactose 2ES enzyme electrode response over the course of a 10-day working life where day 1 is the day of installation.

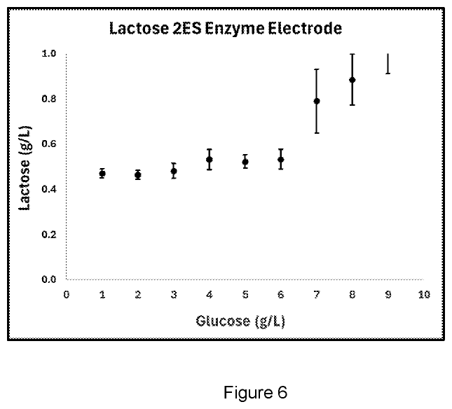

FIG. 6 shows an average response of n=6 lactose 2ES enzyme electrodes to solutions containing a constant concentration of 0.5 g/L lactose with increasing glucose concentrations. Data is generated using a dual chemistry approach where the lactose 2ES enzyme electrode is coupled to a glucose only enzyme electrode. The signal from the glucose electrode is used as a corrective factor for the lactose electrode. This data demonstrates that the lactose response is accurate and stable up to a glucose concentration of approximately 6 g/L.

### FIGS. 7-9

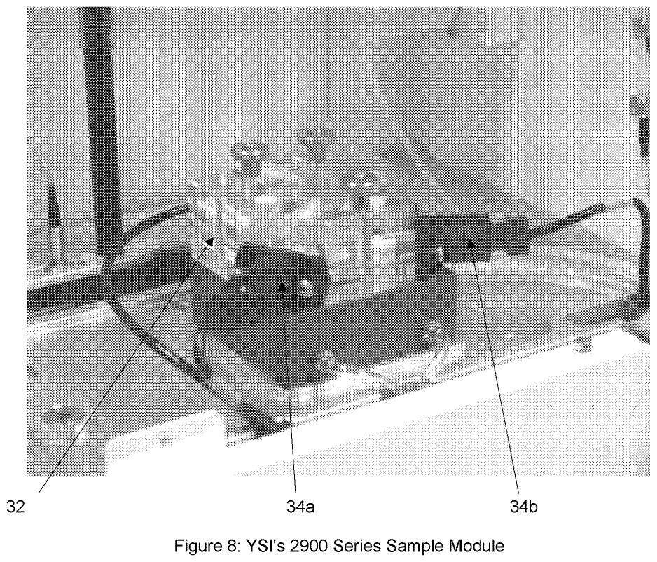

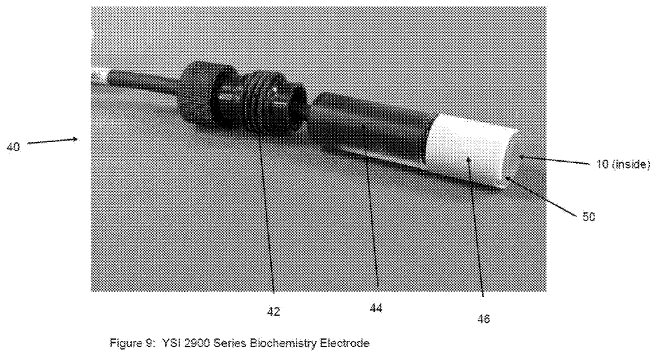

FIG. 7 shows YSI's 2900 Series Biochemistry Analyzer 30 having a sample module 32 (FIG. 8), which forms part of the analyzer 30. The sample module 32 includes three electrodes, two of which are shown and referenced as 34a, 34b. The probes/electrodes 34a, 34b may be configured as YSI's biochemistry electrode shown in FIG. 9. By way of example, the three probes/electrodes may include a lactose enzyme electrode according to the present invention, a glucose enzyme electrode known in the art, and a reference electrode known in the art, e.g., to generate the net lactose signal by subtracting the glucose enzyme electrode signal from the lactose enzyme electrode signal.

FIG. 9 shows YSI's 2900 Series biochemistry electrode generally indicated as 40 having a trilaminar membrane according to the present invention that may be coupled to and form part of YSI's 2900 Series Sample Module 32 shown in FIG. 8. The lactose enzyme electrode 40 includes a retainer 42 and two sleeves 44, 46. The sleeve 46 is configured to receive and contain the laminated membrane 10. The laminated membrane 10 may include an O-ring 50, e.g. affixed to and coupled around an outer surface of the laminated membrane 10 and configured to mount the laminated membrane 10 on the biochemistry electrode or an electrode probe of the sample module 32 of the biochemistry analyzer 30 (FIG. 7). In FIG. 9, the O-ring 50 is affixed to the sleeve 46 as shown. The O-ring 50 enhances the structural integrity of the laminated membrane 10, and also serves to create a water-tight seal between the lactose enzyme electrode 40 and the sample well.

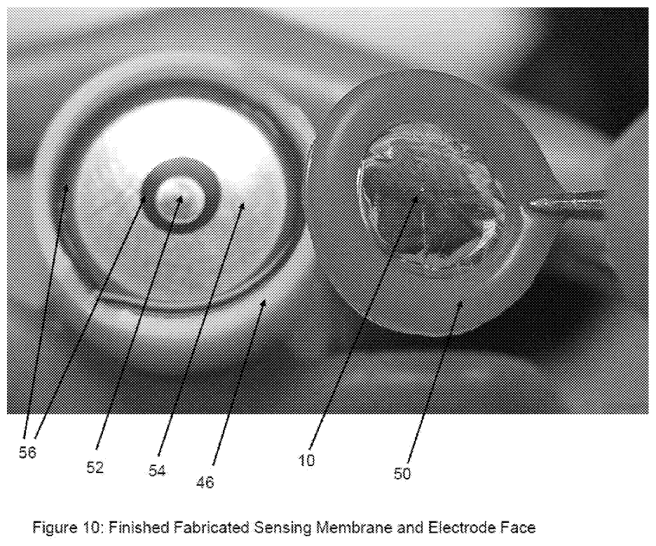

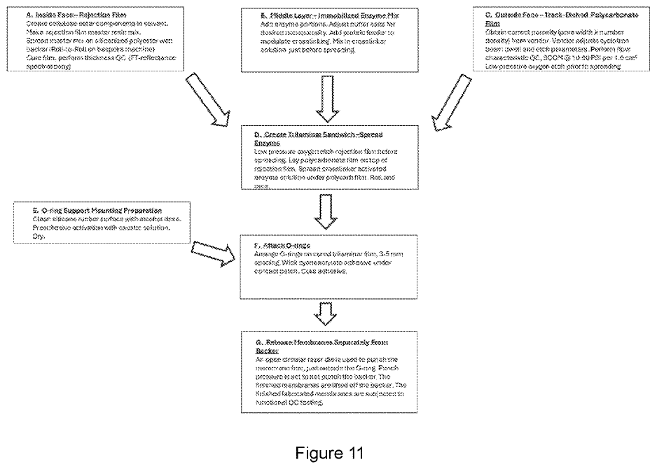

FIG. 10 shows the active face of a YSI 2900 series biochemistry electrode and a finished trilaminar enzyme sensor membrane 10 side by side. The biochemistry electrode includes an operative arrangement of a platinum sense electrode 52, a Ag/AgCl reference electrode 54, an intervening dielectric potting 56, and a plastic outer sleeve 46. Next to the face of the electrode is the trilaminar membrane 10, on the right, mounted to a silicone rubber O-ring 50 gripped by a tweezers and ready for installation onto the electrode face. For scale, the O-ring 50 has a ⅜″ outer diameter.

By way of further example, see YSI's aforementioned U.S. Pat. Nos. 3,539,455; 4,456,074; 4,073,713 and 3,979,274 that disclose embodiments of laminated membranes used together with O-rings. The scope of the invention is not intended to be limited to any particular way of coupling together the O-ring 50 to the outer surface of the laminated membrane 10, e.g., which may include either ways now known in the art or ways later developed in the future. Based upon that disclosed herein, one skilled in the art would appreciate and understand how to couple the laminated membrane 10 together with the O-ring 50 without undo experimentation.

FIG. 11 shows a fabrication process having steps A through G for making trilaminar membranes according to the present invention. By way of example, and in conjunction with that set forth in FIG. 11, the following provides an example of an implementation of the steps A through G of the fabrication process with explicit amounts that was performed during development and led to working membranes:
A. Tested thickness of a cured rejection film is acceptable at 1.5 microns<film <2.5 microns.
B. 2.4 mg of β-Galactosidase (Eschericia coli, grade VIII), 1.0 mg of Glucose Oxidase (Aspergillus niger), 0 mg of BSA as proteic feeder (was not needed) were added to 40 μL of YSI 2357 buffer that had been supplemented to 7 mM MgCl₂. Addition of Succinate and Citrate was done to a combined final molarity of 8.8 mM. Low polymerization glutaraldehyde in the amount of 0.33% was added to effect the crosslinking reaction.
C. The track-etched polycarbonate film used had an areal pore density of 3.0×10⁹ pores/cm² and a flow parameter of 10-20 SCCM/cm². Pore diameter, usually 20-50 nm, was acceptable by virtue of the acceptable flow parameter. Curing of the enzyme trilaminate sheet was done at 45° C. for 30 minutes.
D. As per standard process.
E. As per standard process.
F. Ethyl cyanoacrylate was used, 0.5-2 μL per O-ring. Curing was done at 25-30° C. under a chemical fume extractor for 15 minutes.
G. An automated pneumatic punch with a digital pressure feedback control was used. These membranes were tested on a YSI 2900D Biochemistry Analyzer using a YSI 2357 Buffer. Each 475 mL of buffer (one buffer sachet reconstituted) was supplemented with 5.0 mL of 15% w/v MgCl₂·6H₂O in water solution. Sampling was set to 25 microliters and a 30 second read time was used.

This small R&D scale build yielded 32 sense membranes. The build process scales linearly.

### Other Envisioned Embodiments

By way of further example, the present invention may also take the form of a method for a quantitative determination of a substance which reacts with a 2-enzyme system in a manner whereby a product of the reaction may be analytically measured and wherein the substance is introduced for determination by being included in a buffer solution which is brought into contact with the 2-enzyme system. This method may be suited to and implemented using YSI's Biochemistry Analyzers of the 2700, 2900, and future 29X series in development. The reaction ultimately produces hydrogen peroxide measured amperometrically, e.g., including where the enzyme system may consist of β-galactosidase (beta-galactosidase) and glucose oxidase enzymes.

In particular, the method for the quantitative determination of an amperometrically inactive material which reacts with the enzymes may include the following steps:
(a) providing an electrochemical cell including at least one electrode positioned behind a membrane permeable to the material being measured and in contact with an electrolyte,
(b) establishing a potential across the electrochemical cell such that a current is produced which is proportional to the amount of hydrogen peroxide present on the electrode side of the membrane,
(c) bringing the electrochemical cell into contact with a quantity of material containing amperometrically inactive material in the presence of the enzymes, and
(d) determining the current flowing across the electrochemical cell as an indication of the amount of the substance.

According to some embodiments, the method also may include one or more of the following features:
the enzymes may be immobilized on the membrane; the amperometrically inactive material may be lactose, as well as other analytes now known or later developed in the future;
the determination of the current flowing across the electrochemical cell may be a function of the amount of hydrogen peroxide formed, as well as other by-products now known or later developed in the future;
the enzymes may be immobilized by chemical crosslinking between an inner and outer membrane layer relative to the at least one electrode;
the inner layer may be a cellulose acetate or cellulose acetate butyrate mix and the outer layer, including a track-etched polycarbonate film; and/or
the buffer may contain magnesium ions (Mg 2+) as cofactor, e.g., including in the approximate amount of 0.18 g Mg 2+ per 1000 mL buffer.

### Advantages

Advantages of the present invention may include one or more of the following:

The alternative enzymes are ~⅕ the cost of the current production run and are much more thermostable.

The new laminated membranes may include trilaminar sensing membranes that do not delaminate in production or near-term storage.

The new sensing membranes also do not have co-reactivity to glycerol and galactose, unlike the current membranes.

The two enzymes in the new sensor, β-galactosidase and glucose oxidase, are commodity products.

The key element is that the 2-enzyme system functions equivalently to the known single enzyme galactose oxidase membrane currently used in YSI's Biochemistry Analyzer.

The new lactose membranes can be produced reliably, do not delaminate, and are expected to be produced at a lower cost, resulting in higher profit margins. Critical to this is that the immobilized mixed-enzyme layer has precisely the right enzyme activity and the right molecular porosity to be effective as in YSI's currently used sensing membrane.

This dual enzyme (2ES) sensing membrane is engineered specifically for YSI's biochemistry analyzers for automated potentiostatic amperometry, primarily utilizing YSI's known technology, e.g., including its patents referenced herein and known production methods.

The new sensing membrane using the sequential 2-enzyme system was engineered specifically for YSI's biochemistry analyzers with those specific electrochemical probes.

Another key element is that magnesium ions (Mg²⁺) must be present in the system buffer because it is a required cofactor to the β-galactosidase enzyme. A concentrated cofactor buffer additive can also be developed as a separate product.

β-galactosidase can be obtained as a recombinant protein, derived from many different organisms and with different genetic modifications and properties. The method of enzyme immobilization can be performed using many physicochemical methods.

The use of the 2-enzyme cascade, β-galactosidase then glucose oxidase, to analyze lactose has been in the literature and prior patent applications for many years. YSI has at least 8 patents of prior art in developing membranes for its biochemistry analyzers having a multiplicity of analyte specificities. The new sensing membrane builds on YSI's technology in this regard.

Other advantages include at least the following:
a possible 75% lower production cost,
availability of components by multiple sources (i.e., a non-single source),
greater stability in storage and use, and
an increased profit margin, which is related to the 75% lower production cost.

### Applications

Applications for the present invention may include use on all of YSI's Biochemistry Analyzers, e.g., including its 2700 series, 2500 series and 2900 series and future series in development.

### The Scope of the Invention

While the techniques disclosed herein constitute examples of preferred embodiments of the present invention, it is to be understood that the present invention is not limited to the precise techniques disclosed herein, and that changes may be made thereto without departing from the spirit and scope of the present invention.

1. A laminated membrane to quantitate lactose in an analyte sample using a biochemistry analyzer, comprising:
a support layer configured to control a diffusion rate of an analyte sample having lactose into the laminated membrane;
an exclusion layer configured to diffuse hydrogen peroxide (H₂O₂) from the laminated membrane; and
an enzyme reaction layer having a 2-enzyme system configured between the support layer and the exclusion layer, the 2-enzyme system having β-galactosidase and glucose oxidase configured to process oxidatively the analyte sample having the lactose and produce the hydrogen peroxide (H₂O₂) for diffusing through the exclusion layer and from the laminated membrane and reacting with a sensor electrode to quantitate the lactose in the analyte sample with the biochemistry analyzer.

2. A laminated membrane according to claim 1, wherein the β-galactosidase in the 2-enzyme system is configured to process the analyte sample having the lactose into galactose and β-glucose, and the glucose-oxidase in the 2-enzyme system is configured to process the β-glucose and forms the gluconic acid and hydrogen peroxide (H₂O₂).

3. A laminated membrane according to claim 1, wherein the enzyme reaction layer is configured as an immobilized 2-enzyme layer between the support layer and the exclusion layer; acting as a signal generator, a barrier and an adhesive layer.

4. A laminated membrane according to claim 1, wherein
the 2-enzyme system includes the β-galactosidase and magnesium ions configured to process the lactose into galactose and β-glucose; and
the 2-enzyme system is based upon a reaction, as follows:

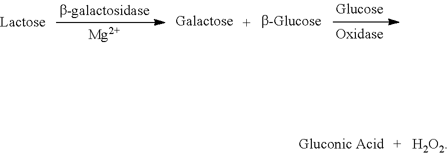

5. A laminated membrane according to claim 1, wherein the support layer is a homogeneous, semi-permeable polycarbonate film, including where the homogeneous, semi-permeable polycarbonate film has a thickness of 5-20 microns and pore sizes of 200 Å or less, with pore densities on an order of 6×10⁸ through 6×10⁹/cm² or some combination thereof.

6. A laminated membrane according to claim 1, wherein the exclusion layer is a film made from a two-component cellulose acetate/cellulose acetate butyrate mixture, including where the film has a thickness less than 3 microns.

7. A laminated membrane according to claim 1, wherein the laminated membrane is configured to, and forms part of, a lactose enzyme electrode.

8. A laminated membrane according to claim 1, wherein the laminated membrane comprises an O-ring affixed to an outer surface of the laminated membrane and configured to mount the laminated membrane on an electrode probe of the biochemistry analyzer.

9. A method to quantitate lactose in an analyte sample using a laminated membrane in a biochemistry analyzer, comprising:
configuring a laminated membrane with a support layer, an exclusion layer and an enzyme reaction layer having a 2-enzyme system with β-galactosidase and glucose oxidase and being arranged between the support layer and the exclusion layer;
controlling with the support layer a diffusion rate of an analyte sample having lactose into the laminated membrane;
restricting interferences with an exclusion layer that also allows diffusion of hydrogen peroxide (H₂O₂) from the enzyme reaction layer of the laminated membrane; and
processing oxidatively with the 2-enzyme system having β-galactosidase and glucose oxidase the analyte sample having the lactose and producing the hydrogen peroxide (H₂O₂) for diffusing through the exclusion layer and from the laminated membrane and reacting with a sensor electrode to quantitate the lactose in the analyte sample using the laminated membrane in a biochemistry analyzer.

10. A method according to claim 9, wherein the method comprises:
processing with the β-galactosidase in the 2-enzyme system the analyte sample having the lactose into galactose and β-glucose; and
processing with the glucose oxidase in the 2-enzyme system the β-glucose and forming gluconic acid and hydrogen peroxide (H₂O₂).

11. A method according to claim 9, wherein the method comprises configuring the enzyme reaction layer as an immobilized 2-enzyme layer to act as a signal generator, a barrier and an adhesive layer.

12. A method according to claim 9, wherein the method comprises configuring the 2-enzyme system with the β-galactosidase and magnesium ions to process the lactose into galactose and β-glucose based upon a reaction, as follows:

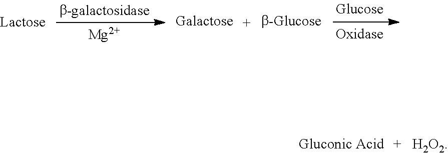

13. A method according to claim 9, wherein the method comprises configuring the support layer as a homogeneous, semi-permeable polycarbonate film, including where the homogeneous, semi-permeable polycarbonate film has a thickness of 5-20 microns and pore sizes of 200 Å or less, with pore densities on an order of 6×10⁸ through 6×10⁹/cm² or some combination thereof.

14. A method according to claim 9, wherein the method comprises configuring the exclusion layer as a film made from a two-component cellulose acetate/cellulose acetate butyrate mixture, including where the film has a thickness less than 3 microns.

15. A method according to claim 9, wherein the method comprises affixing an O-ring to an outer surface of the laminated membrane and mounting the laminated membrane on an electrode probe of the biochemistry analyzer.
